(19) United States
(12) Patent Application Publication
(10) Pub. No.: US 2004/0248205 A1
(43) Pub. Date: Dec. 9, 2004

(54) Major histocompatibility complex (MHC)-peptide arrays

(76) Inventors: Lawrence J. Stern, Acton, MA (US); Jennifer D. Stone, Arlington, MA (US); Gregory J. Carven, Maynard, MA (US); Sriram Chitta, Worcester, MA (US); Iwona Strug, Worcester, MA (US); Jennifer R. Cochran, Philadelphia, PA (US)

Correspondence Address: FISH & RICHARDSON PC, 225 FRANKLIN ST, BOSTON, MA, 02110, US

(21) Appl. No.: 10/823,866

(22) Filed: Apr. 14, 2004

Related U.S. Application Data
(60) Provisional application No. 60/463,379, filed on Apr. 16, 2003.

(51) Int. Cl. G01N 33/53; C12M 1/34

(52) U.S. Cl. 435/7.1; 435/287.2

(57) ABSTRACT

This invention relates to arrays of immobilized major histocompatibility complex (MHC)-peptide complexes and methods for using them.

### CLAIM OF PRIORITY

This application claims priority under 35 USC §119(e) to U.S. Provisional Patent Application Ser. No. 60/463,379, filed on Apr. 16, 2003, the entire contents of which are hereby incorporated by reference.

### FEDERALLY SPONSORED RESEARCH OR DEVELOPMENT

This invention was made with Government support under Grant No. N01-AI95361 awarded by the National Institutes of Health. The Government has certain rights in the invention.

### TECHNICAL FIELD

This invention relates to arrays of immobilized major histocompatibility complex (“MHC”)-peptide complexes and methods for using them.

### BACKGROUND

T cells play a central role in the adaptive immune response to pathogens, both by directly eliminating infected cells (cytotoxicity) and by promoting antibody and inflammatory responses (helper activity). The ability of T cells to recognize and respond to foreign materials is determined by the precise reactivity of their T cell antigen receptors (TCR) to peptides complexed with major histocompatibility complex (MHC) proteins present on the surfaces of other cells (Germain (1994) Cell 76(2):287-99; Davis et al. (1998) Annu. Rev. Immunol. 16:523-44; Hennecke and Wiley (2001) Cell 104(1):1-4; Rudolph and Wilson (2002) Curr. Opin. Immunol. 14(1):52-65). Recognition of a foreign peptide antigen bound to an MHC protein triggers a T cell response that, depending on the T cell lineage and developmental state, can result in T cell activation and proliferation with consequent induction of cytotoxic, inflammatory, suppressor, or effector responses, or in T cell down-modulation, anergy, or death (Qian and Weiss (1997) Curr. Opin. Cell Biol. 9(2):205-12; Cantrell (2002) Immunology 105(4):369-74). Cytotoxicity and helper functions generally are mediated by CD8 and CD4 T cells, respectively, with the relative importance of the two subsets being different in different infections and pathologies. The immune response to pathogens is governed by the distribution within the overall T cell population of both T cell antigen specificities (the T cell repertoire) and T cell effector functions, and by the dynamic responses of that population to the immunological challenge.

Identification of a T cell epitope (the portion of a foreign or self antigen that when bound to an MHC protein, provokes a T cell response) often is a starting point for immunological research into the response of the body against a particular pathogen. Individual differences in MHC haplotype determine which T cell epitopes are presented, and thus play a role in determining individual susceptibility to infection and to effective induction of immunity after vaccination. Knowledge of T cell epitopes can be used to develop subunit and synthetic epitope vaccines, and can help to explain patterns of viral evolution and MHC distribution. Some viral and bacterial pathologies are thought to be linked to cross-reactivity of particular (e.g., pathogenic) T-cell epitopes with homologous self peptides (Baum and Staines (1997) Cytokines Cell. Mol. Ther. 3(2):115-25; Wucherpfennig (2001) J. Autoimmun. 16(3): 293-302; Lang et al. (2002) Nat. Immunol. 3(10):940-3). Finally, several important methods for identifying and tracking antigen-specific T cells, such as MHC tetramer analysis (Altman et al. (1996) Science 274(5284):94-6; Cameron et al. (2002) J. Immunol. Methods 268(1):51-69; Constantin et al. (2002) Biol. Res. Nurs. 4(2):115-27; Kwok et al. (2002) J. Immunol. Methods 268(1):71-81; Xu and Screaton (2002) J. Immunol. Methods 268(1): 21-8), rely on prior identification of the relevant T-cell epitopes.

T cells specific for a particular antigen can express different functional capacities, depending on their lineage, activation state, and history. Analysis of antigen-specific T cells present in mixed populations is an important aspect of immunological research. Successful control of a viral infection requires proliferation of antigen-specific T cell populations derived from naive or memory pools, upregulation of appropriate effector functions, and controlled reduction of the response as the immunological challenge abates (Perelson (2002) Nat. Rev Immunol. 2(1):28-36). To monitor and modulate these processes, immunologists need to identify, enumerate, and functionally characterize antigen-specific T cells present amid a very large excess of T cells of mixed specificities and functions.

Currently, identification of the T-cell epitope(s) recognized by a particular T cell clone or polyclonal population is generally accomplished through testing of a large set of overlapping peptides that span the entire protein of interest. Typically, a cellular readout such as cytokine secretion, proliferation, or cytotoxicity is used to identify positive peptides (Geysen et al. (1984) Proc. Natl. Acad. Sci. U.S.A. 81(13):3998-4002; Rodda (2002) J. Immunol. Methods 267(1):71-7). The large number of peptides to be screened, in many cases several hundreds or even thousands, presents a substantial experimental problem under current methodologies. Pools of several peptides often are assayed together to reduce the number of individual experimental determinations, but this introduces a requirement for additional deconvolution assays to identify the individual peptide(s) responsible for the activity, with a concomitant requirement for additional samples or short-term culture/cryopreservation of the original T cell samples (Van Epps et al. (1999) J. Virol. 73(7): 5301-8; Kwok et al. (2002) supra). Moreover, peptide pool analysis may not be successful in identifying epitopes from highly oligoclonal or polyclonal populations.

Identification of pathogen-derived MHC-binding peptides in principle can facilitate T cell epitope discovery by limiting the number of peptides that need to be evaluated. The basic parameters of MHC-peptide interaction have been elucidated through crystal structures of MHC-peptide complexes, characterization of endogenously bound peptides (Rotzschke et al. (1990) Nature 348(6298): 252-4; Van Bleek and Nathenson (1990) Nature 348(6298):213-6; Hunt et al. (1992) Science 255(5049):1261-3; Reich et al. (1994) J. Immunol. 152(5):2279-88), site-specific mutagenesis (Hammer et al. (1997) Adv. Immunol. 66:67-100), and library screening approaches (Chicz et al. (1992) Nature 358(6389):764-8; Grey et al. (1995) Cancer Surv. 22:37-49; Hammer et al. (1997) supra; Southwood et al. (1998) J. Immunol. 160(7): 3363-73). Allele-specific binding motifs are currently known or predicted for about 100 different MHC proteins (Brusic et al. (1998) Nucleic Acids Res. 26(1):368-71). However, even in the best characterized cases, the binding predictions are woefully inaccurate as guides to T cell epitope identification, with substantial false positive and false negative rates. Computational approaches have not greatly improved this success rate, and experimental characterization of a large set of overlapping peptides containing all possible binding domains remains the norm. Experimental determination of class I and class II MHC peptide binding activity is technically demanding and requires specialized materials for each MHC of interest, only some of which are currently available.

Other methods currently available for detection and functional characterization of T cells specific for a particular antigen in the presence of a much larger number of non-specific T cells include traditional cytotoxicity, cytokine production, and suppressor assays. Limiting dilution analysis or selection of antigen-specific cell lines require extensive in vitro culture, after which relevant T cell phenotypes may be lost, and do not allow for simultaneous testing of multiple antigens or T cell samples. ELISPOT analysis provides a direct measure of functional capacity at the single-cell level, but only cells that secrete a particular cytokine are detected, and responding cells cannot be isolated from the bulk population (Czerkinsky et al. (1983) J. Immunol. Methods 65(1-2):109-21; Carvalho et al. (2001) J. Immunol. Methods 252(1-2):207-18; Mashishi and Gray (2002) Clin. Chem. Lab. Med. 40(9):903-10). Cytokine surface capture, a newly-developed ELISPOT variation, can be used to isolate antigen-specific cells (Mathioudakis et al. (2002) J. Immunol. Methods 260(1-2):37-42). Antibody-based methods for examination of phenotypical analysis of T cell surface expression are well-developed. MHC tetramer analysis, in which fluorescent multimers of specific MHC-peptide complexes are used to directly label antigen-specific T cells, has been used in many aspects of T cell research, but is dependent on production of recombinant MHC-peptide complexes, which are only available for a few alleles. Currently, MHC tetramers are available for less than twenty-five (NIH Tetramer Facility, 2003) of the more than 1500 currently known alleles of conventional class I and II MHC proteins (Robinson et al. (2000) Tissue Antigens. 55(3):280-7; Robinson et al. (2001) Nucleic Acids Res. 29(1):210-3; Robinson et al. (2003) Nucleic Acids Res. 31(1):311-4; and the IMGT/HLA Sequence Database of sequences of the human major histocompatibility complex (HLA), available on the world wide web at ebi.ac.uk/imgt/hla/). This has led to the practice of examining only a very few MHC allotypes, in particular those for which recombinant proteins and MHC tetramers are available, and ignoring the hundreds of other MHC allotypes for which such reagents are not available.

### SUMMARY

The invention is based, in part, on the development of new methods and materials, e.g., arrays, for the rapid identification of T cell epitopes and for the analysis of antigen-specific T cells in human peripheral blood samples. In one aspect, the invention provides arrays, e.g., on “chips,” that are useful, e.g., clinically and experimentally. The arrays can be used in simultaneously screening a number of potential epitopes, e.g., in a high-throughput assay. The arrays can be used to detect the presence of a small number of T cells specific for a given epitope in a large number of other cells, e.g., non-specific T cells, allowing for the detection of exposure to a given antigen or disease. The frequency and nature of T cells responding to a selected epitope can be determined, indicating the type of immune response that is present, e.g., whether an adaptive immune response is present, whether or not an immune response is mounted to a given antigen, or whether the responding populations of T cells are primarily CD4+ or CD8+ cells. Finally, the response to a particular antigen can be evaluated, e.g., to assist in designing or predicting the efficacy of a vaccine.

In one aspect, the invention features an array including a substrate and a plurality of MHC molecules, e.g., two or more, e.g., 12, 24, 36, 48, or 96 to more than 1,000, 2,000 or 3,000, complexed with antigen-derived peptides immobilized on the substrate. The antigen-derived peptides can be any peptide fragment of a selected antigen, e.g., a proteolytic fragment or a synthetically-derived fragment, as described herein. In general, the peptide can be synthetic; the size of the peptide is typically determined by the class of the MHC and can range from 8-, 9-, or 10-mers to 25-mers or more. In one embodiment, the array includes at least about 10, 50, 100, or 1000 different MHC-peptide complexes, e.g., MHC-peptide complexes that differ in peptide sequence or MHC allotype. The MHC molecules can include class I MHC molecules alone, class II MHC molecules alone, or both class I and class II MHC molecules.

In some embodiments, spatially-distinct areas are surrounded by a hydrophobic barrier, either individually or in groups (e.g., all surrounded by a single barrier). In some embodiments, the substrate is optically transparent. In other embodiments, the substrate is opaque, e.g., black. The substrate can be glass, quartz, polystyrene, polycarbonate, polypropylene, polymethacrylate, silicon, or another polymer or plastic. The substrate can be essentially two-dimensional, e.g., a flat array on a slide or coverslip, and the MHC-peptide complexes can be immobilized in spatially-distinct, individually addressable areas, or the substrate can be amorphous, e.g., a flowmetric array or quantum dot array as described herein, and the MHC-peptide complexes are immobilized or bound to differentially addressable beads or quantum dots. The substrate can be coated with gold, biotin streptavidin, or another molecule suitable for immobilizing the MHC molecules.

In some embodiments, the MHC-peptide complexes are immobilized on the substrate via the MHC molecules, which are immobilized on the substrate using methods known in the art, e.g., via direct adsorption; peptide linkers; biotin-streptavidin; cysteine attachment, e.g., via disulfide, maleimide, vinyl sulfone, or haloacetyl chemistries; amine attachment, e.g., via N-hydroxy succinimide or other activated esters; metal chelate interaction, e.g., via hexahistidine or other poly-histidine tags and immobilized metal ion such as nickel-ntirotrilotracetic acid; or other covalent or non-covalent attachment chemistries known in the art.

In some embodiments, the MHC-peptide complexes are immobilized on the substrate via the peptides can be immobilized, e.g., through biotin-mediated immobilization to a streptavidin-coated surface using a long, flexible linker, and soluble MHC proteins may be bound to the peptides in a way such that T cells can access the relevant binding surface. In other embodiments, the MHC-peptide complexes are immobilized on the substrate via the antigen-derived peptide, which is immobilized on the surface using methods known in the art, e.g., via direct adsorption; peptide linkers; biotin-streptavidin; cysteine attachment, e.g., via disulfide, maleimide, vinyl sulfone, or haloacetyl chemistries; amine attachment, e.g., via N-hydroxy succinimide or other activated esters; metal chelate interaction, e.g., via hexahistidine or other poly-histidine tags and immobilized metal ion such as nickel-ntirotrilotracetic acid; or other covalent or non-covalent attachment chemistries known in the art. In some embodiments, the complexes are bound by immobilizing a biotinylated peptide on a streptavidin-coated surface. In some embodiments, the array is created by arraying the peptides alone, followed by loading of the array with soluble MHC proteins, which become immobilized by their interaction with the arrayed peptides. The soluble MHC proteins can be provided “empty,” i.e., without any bound peptide, or as complexes with an easily exchanged peptide, e.g., a peptide that binds with very low affinity or that rapidly exchanges, e.g., as are known in the art. In some embodiments, the MHC-peptide complexes are preformed, and the complete complexes are immobilized on the array via the peptide.

In certain embodiments, the array also includes costimulatory molecules (e.g., molecules that have costimulatory activity) immobilized along with the MHC-peptide complexes. In some embodiments, the costimulatory molecules include costimulatory antibodies, e.g., anti-CD2, anti-CD11a, anti-CD28 and/or anti-CD49d, and costimulatory agents, e.g., molecules, other than antibodies, that have costimulatory activity, such as B7-1 (also known as CD80) B7-2 (also known as CD86), ICOSL (also known as B7h, LICOS, B7-H2, or B7RP-1), B7-H1 (also known as PD-L1), B7-DC (also known as PD-L2), B7-H3, B7-H4, or other member of the B7 family (see, e.g., Rietz and Chen, (2004) Am. J. Transpl. 4:8-14), LFA-3, and/or ICAM-1, ICAM-2, or other members of the ICAM family.

In some embodiments, the array also includes anti-factor antibodies immobilized with the MHC-peptide complexes. In some embodiments, the antibodies are specific for a factor secreted or expressed by activated T cells, e.g., antibodies specific for cytokines or other factors secreted by activated T cells, e.g., one or more antibodies specific for IL-2, IL-3, IL-4, IL-5, IL-6, IL-7, IL-9, IL-10, IL-12, IL-13, IL-16, interferon-gamma (IFN-γ), tumor necrosis factor alpha (TNF-α), and TNF-β, GM-CSF, oncostatin M (OM, OSM), macrophage migration inhibitory factor (MIF), TNF-Related Apoptosis Inducing Ligand (TRAIL, also known as Apo-2L), 4-1BB ligand (4-1BBL, also known as CD137 ligand or TNFSF9), perforins including cytolysin (Ishikawa et al., J. Immunol. 143:3069-3073 (1989)), granzymes (e.g., granzyme A or B), and/or alpha-defensin (see, e.g., Janeway et al., Immunobiology, Austin and Lawrence, Eds. (Garland Publishing, New York, 2001)). A number of suitable antibodies, and methods for making them, are known in the art. See, e.g., Antibodies: A Laboratory Manual, Harlow and Lane, eds. (Cold Spring Harbor Laboratory, Cold Spring Harbor, N.Y. 1988). Although the term “cytokine capture” is used herein, it is not intended to be limited to classic cytokines but can include any factors secreted by activated T cells.

In some embodiments, the array includes both anti-factor antibodies and costimulatory molecules immobilized along with the MHC-peptide complexes.

In another aspect, the invention features methods of making an array as described herein.

In another aspect, the invention features methods for identifying a T cell epitope. The methods can include providing an array including a substrate and a plurality of MHC molecules complexed with antigen-derived peptides, immobilized on the substrate as described herein; contacting the array with a sample comprising T cells; and detecting a T cell interaction with an epitope. The T cell epitope is identified based on the identity of the MHC-peptide complex (e.g., the sequence of the antigen-derived peptide, or the class or specific allele of the MHC). The interaction can be detected by detecting activation of T cells, e.g., by secretion or changes in expression of cytokines or other molecules, e.g., IL-2, IL-3, IL-4, IL-5, IL-6, IL-7, IL-9, IL-10, IL-12, IL-13, IL-16, interferon-gamma (IFN-γ), tumor necrosis factor alpha (TNF-α), and TNF-β, GM-CSF, oncostatin M (OM, OSM), macrophage migration inhibitory factor (MIF), TNF-Related Apoptosis Inducing Ligand (TRAIL, also known as Apo-2L), 4-1BB ligand (4-1BBL, also known as CD137 ligand or TNFSF9), CD30 ligand (CD30L), CD27 ligand (CD27L), Fas ligand (FasL), CD40 ligand (CD40L), and/or alpha-defensin (Jia et al., 2000 Gene 263:211-218), changes in the expression levels e.g., an increase or decrease in expression levels, of activation or adhesion markers, e.g., CD3, CD4, CD8, CD11a, CD25, CD27, CD28, CD44, CD49e, CD62L, CD69, CD71, CD95, CD152, and/or Ly6A, and/or by changes in intracellular signals, e.g., calcium flux, or calcium, serine or threonine phosphorylation.

The invention has a number of advantages. Current approaches to investigation of the human cellular immune response suffer from several deficiencies, which are remedied in the invention. In current methods, significant effort is required to identify T cell epitopes, and the low throughput and serial nature of current assay technology hinders the evaluation of T cell function for the small numbers of cells present in clinical samples. Because of these limitations, many aspects of the human immune response, e.g., responses to biodefense-related pathogens, are unknown. For example, in many cases, the relevant T cell epitopes have not been identified, relevant T cell effector functions have not been characterized, and progress in understanding disease pathogenesis and developing vaccines is slow. The present invention provides sensitive, automatable detection methods that can be used in virtually any size assay, e.g., in small volume assays or in high-throughput assays. A further advantage of the MHC-peptide arrays of the invention over current methodologies for identifying T cell epitopes and detecting antigen-specific T cells (such as ELISPOT analysis and MHC tetramer staining) is that many potential antigens can be evaluated in parallel using a small T cell sample, generally without requiring further deconvolution.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic diagram of an immobilized MHC array.

FIG. 2A is a series of photographs representing three successively magnified views of a high-density array of HLA-DR1 MHC molecules on a standard one inch glass slide, at a density corresponding to approximately 3,000 spots per slide. DR1 was detected by Cy-5 fluorescent labels incorporated through amines on the surface of the MHC molecules. Increasing concentrations of DR1-Cy5 resulted in increasing fluorescent intensity, indicated by a shift in pseudocolor from blue, through green and yellow to almost white at the highest intensity spots.

FIG. 2B is a series of photographs of low density arrays of unlabelled HLA-DR1 MHC molecules, spotted manually onto glass (top panel) and plastic-coated (bottom panel) slides. DR1 was detected by the conformation-specific anti-DR antibody LB3.1, labeled with Cy-s, indicating that the protein remains native. These pseudocolored images show increasing fluorescent intensity by a shift from blue, through green and yellow to red.

FIG. 2C is a photograph of a low-density array of murine K^{b}SIY and K^{b}OVA, either native or boiled, spotted manually onto a plastic-coated slide. Detection was effected using biotinylated, conformation-specific antibodies and Cy5-streptavidin. Native, but not boiled, proteins were detected (lighter blue areas), indicating that the native conformation survives the arraying procedure.

FIGS. 3A-3D are schematic diagrams illustrating possible T cell detection methods. In each panel, the specific MHC-peptide complex on the left induces activation, while the non-specific complex on the right does not. FIG. 3A illustrates a method wherein T cell adhesion/binding can be visualized after washing away non-adhering cells. FIG. 3B illustrates a method wherein T cell activation marker upregulation is detected using labeled antibodies, e.g., fluorescently-labeled antibodies. FIG. 3C illustrates a method wherein intracellular signals, e.g., calcium flux, can be visualized using fluorescent dyes. FIG. 3D illustrates a method wherein cytokines or other factors secreted by activated T cells can be captured via a co-immobilized antibody, and detected using a secondary antibody specific for the cytokines or other factors, e.g., the cytokine capture technique.

FIG. 4 is a photograph of a polystyrene slide, manually spotted with MHC-peptide complexes and antibody. The darker areas around the perimeter of the slide are a hydrophobic barrier that allows the entire slide to be incubated with small volumes of T cell-containing solutions, allowing for the use of minimal volumes of T cell-containing solutions while studying a large number of peptide complexes.

FIG. 5A is a line graph showing activation of SCOZA 3.12 polyclonal human CD8+ T cells (specific for HLA-A2/VAC165 pep) by immobilized HLA-A2, as evidenced by CD25 upregulation detected by FITC-conjugated antibody staining. CD69 was similarly upregulated.

FIG. 5B is a line graph showing activation of HA 1.7 human CD4+ T cell clones (specific for HLA-DR1/Ha pep) by immobilized HLA-DR1, as evidenced by IFNγ secretion as measured by ELISA. Immobilized monomers and tetramers both cause activation, with tetramers being about 1,000-fold more potent.

FIG. 6 is a photograph of an MHC array after incubation with SCOZA 3.12, a polyclonal human CD8+ T cell line specific for HLA-A2/VAC165, developed for IFNγ secretion. The left section of the array is immobilized α-CD3, the middle section is immobilized A2-74A (non-specific MHC peptide complex), and the right section is immobilized A2-165 (specific MHC-peptide complex). Columns within each section represent different combinations of co-immobilized costimulatory molecules.

FIG. 7A is a schematic spotting map of biotinylated HLA-DR1 complexes tetramerized with streptavidin and immobilized by adsorption onto a polystyrene slide. **Empty means no peptide is loaded in the peptide-binding groove of HLA-DR1.

FIG. 7B is a fluorescence image of an array, showing detection of native HLA-DR1 by LB3.1-Cy3. Each positive area on this pseudocolored image shows increasing fluorescent intensity by a shift from blue, through green and yellow to red.

FIG. 7C is a fluorescence image of an array, showing detection of secreted mouse IL2 by sandwich ELISA with biotinylated anti-mouse IL2 pre-incubated with streptavidin-Alexa 647.

### DETAILED DESCRIPTION

The present invention provides methods and devices for the simultaneous investigation of numerous, different T cell epitopes. For example, the invention provides methods for understanding host-pathogen interactions, mechanisms of viral and bacterial evasion of the immune response, and individual differences in immune response to infection and vaccination, as well as methods for developing and evaluating therapeutic agents for use in modulating, e.g., enhancing or suppressing, the immune response, e.g., to a particular pathogen. The invention also provides methods for evaluating candidate vaccines, which is particularly useful in human studies where experimental pathogenic challenge is not appropriate.

The new methods and materials are useful to identify T cell epitopes and T cell responses to certain agents. The new technology can also be used to track vaccine effectiveness and test for exposure to certain diseases in clinical samples. The frequency and nature of cells responding to certain epitopes can be quantified, which can indicate that an adaptive immune response is present for a given infection. This capability is valuable to hospitals or other medical centers.

Samples from a typical study of vaccine efficacy might consist of 5-50 ml of peripheral blood (about 5-75 million T cells) from subjects immunized with a vaccine. The T cells of interest are likely to be present at relatively low frequency (e.g., 10⁻³ to 10⁻⁶) within these samples, necessitating examination of a large number of cells to find a significant number of responders. However, in many cases a large number of potential antigens (e.g., 10² to 10⁴) needs to be evaluated using these samples. The invention provides sensitive methods and devices for evaluating large numbers of potential antigens that require only a small sample size, compatible with clinical samples. Thus, facile identification of T cell epitopes and the routine enumeration and functional analysis of antigen-specific T-cells in clinical samples is possible using the methods and devices described herein. The invention provides new capabilities to the general immunological community in their efforts to understand and manipulate the human immune response, e.g., to autoimmune or biodefense-related pathogens.

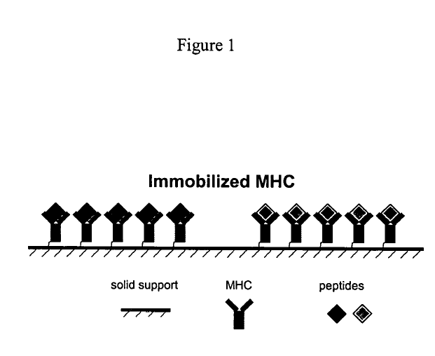

In one aspect, the invention provides arrays, e.g., immobilized arrays of MHC molecules, peptides, and/or MHC-peptide complexes. For example, MHC-peptide complexes are immobilized in one or more spatially-distinct areas of a solid substrate using standard arraying technology, to produce immobilized MHC arrays (see FIG. 1). In some embodiments, each area of the array can carry one or more MHC-peptide complexes that are different from the complexes in at least one other area of the array. In some embodiments, each area or areas of the array can carry a different MHC-peptide complex, or each area or areas can carry a set of the same complexes, with each area or areas having a different type of complex. The MHC-peptide complexes can include a set of MHC molecules chosen for their prevalence, e.g., in the general population, or on a specific subpopulation, e.g., a subpopulation based on ethnic origin, or based on an individual's specific MHC haplotype.

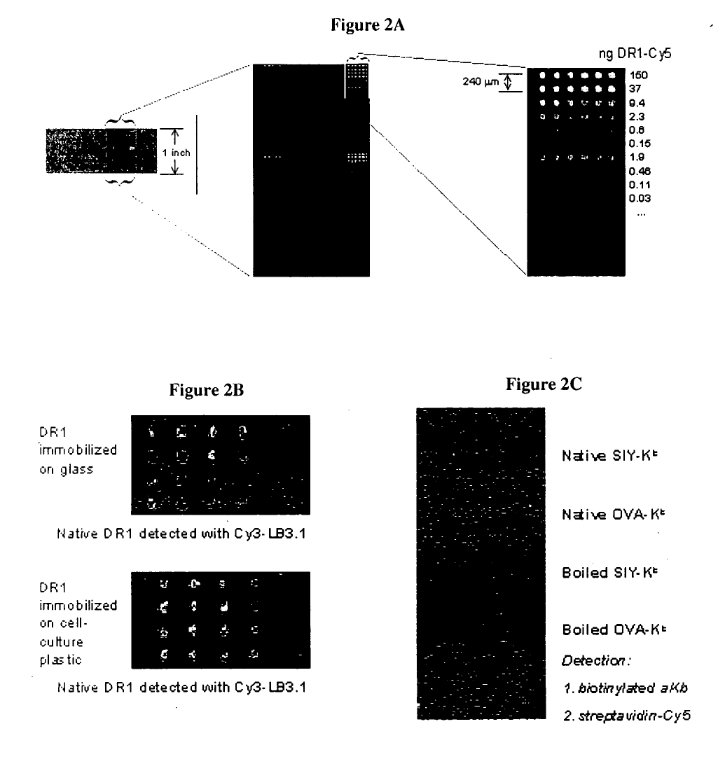

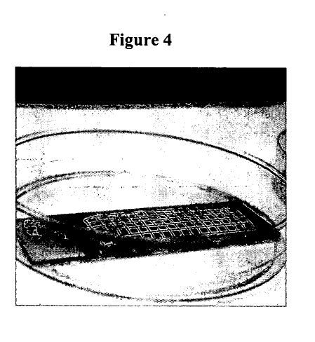

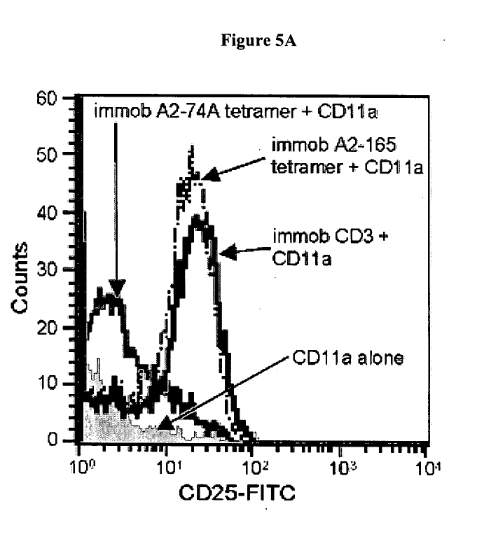

In some embodiments, all potential epitopes for a given protein antigen can be evaluated as sets of overlapping peptides (bound to MHC molecules). Methods for prediction or measurement of peptide binding, e.g., computer-based methods, can be used to provide a narrower set of peptide-MHC complexes derived from a given protein antigen. A variety of solid substrates and immobilization chemistries can be used for the array. For example, standard 2 cm ×2 cm square (e.g., coverslips) or 1 inch×3 inch rectangular (e.g., slides) formats can be used as solid substrates for compatibility with standard array readers, but other formats can also be used. In some embodiments, the MHC-peptide complexes can be immobilized by direct adsorption, by streptavidin-capture, or through a covalent attachment strategy. From 12, 24, 48, or 96 to more than 1,000, 2,000 or 3,000 MHC-peptide complexes can be immobilized in native form in spatially-distinct areas of a solid substrate (see FIGS. 2A-D). Hydrophobic barriers can be incorporated into the slide format and hydrated chambers allow full coverage of an entire slide, e.g., with an approximately 1 ml sample (see FIG. 4).

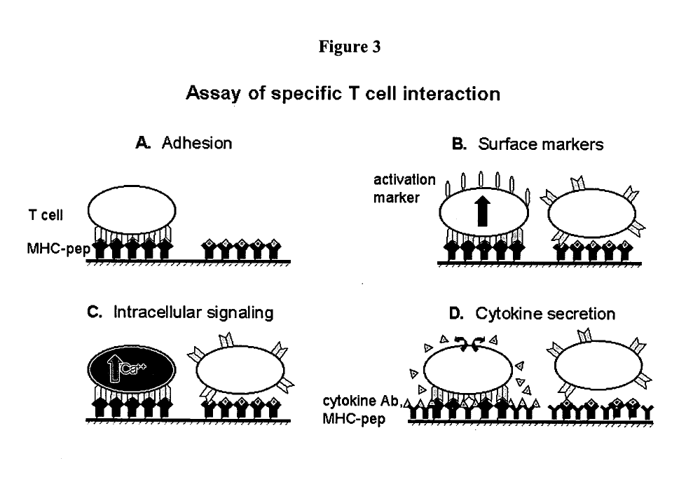

After immobilization of MHC-peptide complexes and appropriate blocking of non-specific sites using buffered solutions of proteins, detergents, or other mixtures conventionally used to bind non-specific binding sites in solid phase assays, e.g., 5% bovine serum albumin, 2% casein, 0.01% gelatin, 0.1% TweenT™-20, 10% serum, or 2% non-fat dried milk, the devices can be incubated, e.g., with purified T cells or peripheral blood mononuclear cell (PBMC) preparations. Immobilized peptide-MHC complexes can activate several responses in T cells, such as calcium flux, downregulation of T cell receptor complex, upregulation of activation markers on the cell surface such as CD69, CD25, and CD71, and secretion of factors including cytokines such as IL2 and IFNγ; any of these, or any other detectable response, can serve as a readout for identifying positive responses (see FIGS. 3A-D and 5A-B).

Various approaches can be employed to detect productive T cell responses to the immobilized stimuli, including, but not limited to, direct binding assays, activation of Ca⁺⁺ transients, up-regulation and down-regulation of T-cell surface markers, and induction of secretion of factors such as cytokines. Sensitivity limits can be estimated, e.g., by dilution of antigen-specific T cells into mixtures of non-specific peripheral blood mononuclear cells (PBMC).

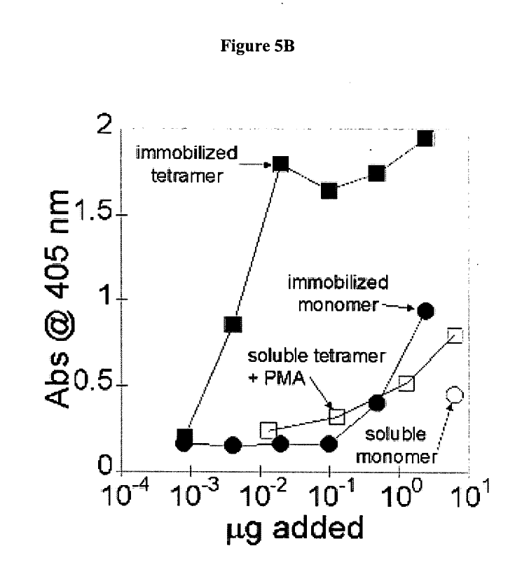

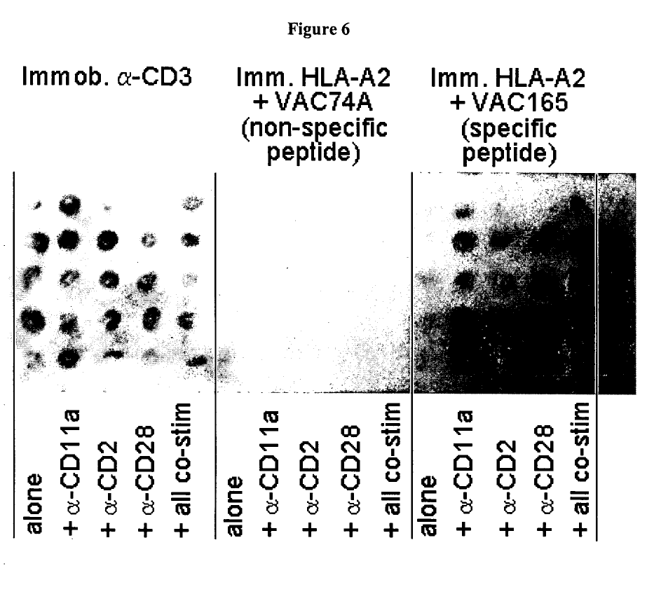

Co-immobilization of adhesion and/or costimulatory molecules can be used to promote productive MHC/T-cell interaction. In some embodiments, a variety of co-immobilized antibodies and purified proteins can be used to provide the necessary costimulation required to elicit certain activation markers. The data (FIGS. 5A-B and 6) and published results (Zaru et al. (1999) J. Immunol. 168(9): 4287-91) suggest that several T cell activation markers are induced by interaction with immobilized MHC-peptide complexes in the presence of such costimulation. For example, vaccinia and influenza virus-specific CD8+ and CD4+ T cells can be detected using immobilized MHC-peptide complexes with co-immobilized CD11a, and a cytokine-capture strategy (see FIG. 6). Peptide-specific T cell interaction only in the expected array areas, even with extended incubation (72 hours), suggests that peptide exchange will not be a problem for many complexes.

Methods of Making MHC-Peptide Arrays

Design of MHC-Peptide Arrays

The MHC-peptide arrays of the invention can consist of any suitable substrate, e.g., chips, e.g., plastic, glass, or gold, including but not limited to polystyrene cell culture slides, 25 mm×75 mm, one end frosted (e.g., Nalge Nunc International) and pre-cleaned glass microslides, 25 mm×75 mm, one end frosted (e.g., VWR).

The MHC chips can also have a surrounding hydrophobic barrier, e.g., to allow incubation of all array elements simultaneously with a small volume of T-cell containing medium. The barrier can be applied using any method known in the art, including by hand or by machine, e.g., using an automated machine, or any commercially available printing method. The hydrophobic barrier can comprise any suitable material, e.g., hydrophobic ink (e.g., Super Pap Pen Liquid Blocker hydrophobic ink (Ted Pella, Inc.)) or silicone (e.g., Press-to-Seal silicone isolator with adhesive, 24 wells, 2.5 mm diameter, 2.0 mm deep (Molecular Probes) or Press-to-Seal silicone sheet with adhesive, 13 cm×18 cm, 0.5 mm thick (Molecular Probes)).

In addition to the active MHC-peptide antigen-containing areas, MHC chips can carry standards or controls, e.g., calibration and positive and negative standards, as well as identifiers, e.g., barcodes or other markings suitable for machine reading.

In some embodiments, each area of the array can carry an MHC-peptide complex that is different from at least one other area of the array, e.g., differs in peptide sequence or MHC allotype. In some embodiments, each area of the array can carry a different MHC-peptide complex (or set of the same complexes in each area). In some embodiments, each area carries the same MHC molecule as every other area, but at least some of the areas carry different peptides from some of the other areas. The array can include regions of redundancy, e.g., a set of areas can carry the same MHC-peptide complexes, or set of MHC-peptide complexes, as another set of areas. In some embodiments, all of the peptides are derived from the same antigen. In some embodiments, all of the peptides are the same. In some embodiments, the array includes one or more sets of areas, wherein all of the areas in the set carry the same MHC molecule and/or the same peptide. In some embodiments, the array includes antigen-derived peptides possible from a single antigen, e.g., an antigenic protein. In some embodiments, the array includes all of the peptides that can be derived from a single antigen. In some embodiments, the array includes peptides derived from more than one antigen, e.g., all of the proteins encoded by a viral genome.

In some embodiments, where there is a need to detect T cells, given current detection technology, an upper limit of density can be roughly estimated as follows: a detection limit of approximately 100 T cells (˜5 micron diameter) corresponds to an array element approximately 50 micron square, approximately 1600 of which can be accommodated in each of the 2×2 cm areas on a standard format slide, allowing for approximately 3200 areas per slide maximum.

In other embodiments, the MHC-peptide complexes can be attached to the surface of differentially addressable beads, e.g., polymeric beads, e.g., polystyrene, e.g., bead arrays, for example, as described in U.S. Pat. No. 5,800,992. The beads correspond to the individually addressable areas in the flat arrays described herein, and can be assayed using, e.g., standard flow metric techniques, e.g., as described in Fulton (1997) Clin. Chem., 43:1749-1756, or other methods as described herein and known in the art.

In other embodiments, the MHC-peptide complexes can be assayed as a quantum dot array, e.g., MHC-peptide complexes attached to quantum dots in a suitable ratio (see, e.g., Watson et al., BioTechniques, 2003; 34(2):296-300, 302-3; Goldman et al., J. Am. Chem. Soc. 2002;124(22):6378-82; Han et al., Nat. Biotechnol. 2001;19(7):631-5; Chan et al., Science 1998;281(5385):2016-8), and assayed using flow cytometry or other assays described herein. For example, a number of complexes can be attached to a single quantum dot, or a number of dots can be attached to a single complex. Multiple complexes can also be attached to multiple linked dots. In one embodiment, each different MHC-peptide complex type (e.g., including a unique peptide or unique MHC allotype) is associated with quantum dots having unique fluorescence properties.

Co-Immobilized Costimulatory Molecules

In some embodiments, each area of the array can also carry immobilized costimulatory molecules, e.g., to allow induction of certain T cell responses, e.g., T cell proliferation or secretion of IL-2 and/or other factors, that may not be induced efficiently by interaction with the immobilized MHC in the absence of such costimulation. Such costimulatory molecules can include one or more costimulatory antibodies, such as anti-CD2, anti-CD11a, anti-CD28, and/or anti-CD49d. The costimulatory molecules can also include one or more soluble costimulatory ligands such as B7-1 (also known as CD80) B7-2 (also known as CD86), ICOSL (also known as B7h, LICOS, B7-H2, or B7RP-1), B7-H1 (also known as PD-L1), B7-DC (also known as PD-L-2), B7-H3, B7-H4, or other member of the B7 family (see, e.g., Rietz and Chen, (2004) Am. J. Transpl. 4:8-14), LFA-3, and/or ICAM-1, ICAM-2, or other members of the ICAM family, which are immobilized on the surface using methods known in the art, e.g., via direct adsorption; peptide linkers; biotin-streptavidin; cysteine attachment, e.g., via disulfide, maleimide, vinyl sulfone, or haloacetyl chemistries; amine attachment, e.g., via N-hydroxy succinimide or other activated esters; metal chelate interaction, e.g., via hexahistidine or other poly-histidine tags and immobilized metal ion such as nickel-ntirotrilotracetic acid; or other covalent or non-covalent attachment chemistries known in the art. The costimulatory molecules can also include other costimulatory agents, e.g., agents that boost the T cell response, such as synthetic analogs or active fragments of natural costimulatory molecules, e.g., B7-1 (also known as CD80) B7-2 (also known as CD86), ICOSL (also known as B7h, LICOS, B7-H2, or B7RP-1), B7-H1 (also known as PD-L1), B7-DC (also known as PD-L2), B7-H3, B7-H4, or other member of the B7 family (see, e.g., Rietz and Chen, (2004) Am. J. Transpl. 4:8-14), LFA-3, and/or ICAM-1, ICAM-2, or other members of the ICAM family, e.g., provided as soluble fragments or fusion proteins, e.g., fused with Ig molecules.

Cytokine Capture Arrays

In some embodiments, each area of the array also includes immobilized antibodies, e.g., one or more antibodies specific for a factor secreted by activated T cells, e.g., IL-2, IL-3, IL-4, IL-5, IL-6, IL-7, IL-9, IL10, IL-12, IL-13, IL-16, interferon-gamma (IFN-γ), tumor necrosis factor alpha (TNF-α), and TNF-β, GM-CSF, oncostatin M (OM, OSM), macrophage migration inhibitory factor (MIF), TNF-Related Apoptosis Inducing Ligand (TRAIL, also known as Apo-2L), 4-1BB ligand (4-1BBL, also known as CD137 ligand or TNFSF9), perforins including cytolysin (Ishikawa et al., J. Immunol. 143:3069-3073 (1989)), granzymes (e.g., granzyme A or B) and/or alpha-defensin (see, e.g., Janeway et al., Immunobiology, Austin and Lawrence, Eds. (Garland Publishing, New York, 2001. Although the term “cytokine capture” is used herein, it is not intended to be limited to classic cytokines but can include any factors secreted by activated T cells. These antibodies can be used to detect secretion of one or more specific secreted factors; for example, the cells can be washed off and binding of factors to the antibodies can be detected using methods known in the art, e.g., as described herein. The antibodies are immobilized on the surface using methods known in the art, e.g., via direct adsorption; peptide linkers; biotin-streptavidin; cysteine attachment, e.g., via disulfide, maleimide, vinyl sulfone, or haloacetyl chemistries; amine attachment, e.g., via N-hydroxy succinimide or other activated esters; metal chelate interaction, e.g., via hexahistidine or other poly-histidine tags and immobilized metal ion such as nickel-ntirotrilotracetic acid; or other covalent or non-covalent attachment chemistries known in the art.

A number of suitable antibodies are known in the art and can be obtained, e.g., commercially. Alternatively, methods for generating such antibodies are known in the art, see, e.g., Liddell and Cryer, A Practical Guide to Monoclonal Antibodies, (John Wiley and Sons, New York, 1991); Monoclonal Antibodies: A Practical Approach, Shepherd, Shepherd and Dean (Editors) (Oxford University Press, Oxford, UK 2000). Briefly, monoclonal antibodies can be generated by immunizing a subject with an immunogen. At the appropriate time after immunization, e.g., when the antibody titers are at a sufficiently high level, antibody producing cells can be harvested from an immunized animal and used to prepare monoclonal antibodies using standard techniques. For example, the antibody producing cells can be fused by standard somatic cell fusion procedures with immortalizing cells to yield hybridoma cells. Such techniques are well known in the art, and include, for example, the hybridoma technique as originally developed by Kohler and Milstein, (1975) Nature, 256: 495-497), the human B cell hybridoma technique (Kozbar et al., (1983) Immunology Today, 4:72), and the EBV-hybridoma technique to produce human monoclonal antibodies (Cole et al., (1985) Monoclonal Antibodies and Cancer Therapy, Alan R. Liss, Inc. pp. 77-96). Technology for producing monoclonal antibody hybridomas is known in the art.

Monoclonal antibodies can also be made by harvesting antibody producing cells, e.g., splenocytes, from transgenic mice expressing human immunoglobulin genes and which have been immunized with monosialo-GM2. The splenocytes can be immortalized through fusion with human myelomas or through transformation with Epstein-Barr virus (EBV). These hybridomas can be made using human B cell-or EBV-hybridoma techniques described in the art (see, e.g., Boyle et al., European Patent Publication No. 0 614 984).

Hybridoma cells producing a monoclonal antibody which specifically binds to monosialo-GM2 are detected by screening the hybridoma culture supernatants by, for example, screening to select antibodies that specifically bind to the immobilized monosialo-GM2, or by testing the antibodies as described herein to determine if the antibodies have the desired characteristics, e.g., the ability to inhibit cell proliferation.

Hybridoma cells that produce monoclonal antibodies that test positive in the screening assays described herein can be cultured in a nutrient medium under conditions and for a time sufficient to allow the hybridoma cells to secrete the monoclonal antibodies into the culture medium, to thereby produce whole antibodies. Tissue culture techniques and culture media suitable for hybridoma cells are generally described in the art (see, e.g., R. H. Kenneth, in Monoclonal Antibodies: A New Dimension In Biological Analyses, Plenum Publishing Corp., New York, N.Y. (1980). Conditioned hybridoma culture supernatant containing the antibody can then be collected.

Production of Recombinant MHCs

Class I and class II MHC proteins can be produced in recombinant form, e.g., as soluble purified proteins, with or without C-terminal biotin tails for immobilization to streptavidin-coated surfaces. Suitable methods for purifying MHC proteins include standard immunobead-purification techniques or chromatographic separation, e.g., as described in Ferre et al., (2003) Protein Sci. 12(3):551-9. Endogenous detergent-solubilized MHC proteins (Gorga et al. (1987) J. Biol. Chem. 262(33):16087-94) can also be used and incorporated into lipid bilayer-coated array substrates by detergent dilution using methods known in the art.

Purification of Recombinant MHC

Peptide complexes of class I and class II MHC proteins can be produced as soluble purified proteins using art-known methods (Class I MHC: Garboczi et al., Proc. Natl. Acad. Sci. USA 89, 3429-3433 (1992); Class II MHC: Frayser et al., Protein Expr. Purif. 15:105-114 (1999); Stern and Wiley, Cell 68:465-477 (1992)). In general, class I MHC alpha and beta subunits can be produced in E. coli as inclusion body products, then purified from the inclusion bodies and refolded, with or without a selected peptide or population of peptides. In general, class II MHC can be produced in insect cells; MHC produced in insect cells are properly folded and are “unloaded,” e.g., are not complexed with a peptide. In some embodiments, the MHC are produced including at least one cysteine residue useful for attaching the MHC to the substrate.

Biotinylation of Recombinant MHC

Recombinant MHC proteins can be biotinylated at an introduced cysteine residue using an thiol-specific biotinylation reagent such as such a biotin-maleimide (Sigma) (Cameron et al, J. Immunol. Methods, 268(1):51-69 (2002)). C-terminal biotin tails can be added to recombinant MHC for immobilization to streptavidin-coated surfaces using art-known methods (Chapman-Smith and Cronan, Biomol. Eng. 16, 119-125. (1999); Altman et al., Science 274, 94-96 (1996); Cameron et al, J. Immunol. Methods, 268(1):51-69 (2002)).

Preparation of Antigen-Derived Peptides

Antigen-derived peptides can be made using methods known in the art, including but not limited to synthesis, e.g., robotic synthesis using FMOC chemistry with DCC coupling and Rink synthesis resins, or digestion of a selected antigen and purification of the resulting peptides.

Refolding Matrix Assay

To evaluate whether a selected peptide will bind to a particular MHC, its ability to promote refolding in an in vitro refolding assay can be determined. Peptides that bind the MHC will allow the MHC to refold, producing conformationally correct proteins, while peptides that do not bind the MHC do not promote refolding; thus, the presence of correctly folded MHC molecules can be used to identify peptides that bind the MHCs (Ostergaard Pedersen et al., Eur. J. Immunol. 31: 2986-2996 (2001); Ferre et al. Protein Science 12:551-559 (2003)). Briefly, urea-solubilized MHC class I or II subunits can be diluted, e.g., into microtiter or “deepwell” 96-well plates containing peptides and incubated, e.g., at 4-14° C., until sufficient refolding has occurred. Refolded proteins can be detected, e.g., using a conformationally-specific, pan-class I antibody such as W6/32 to detect properly refolded MHC I molecules (Maziarz et al., Immunogenetics 24(3):206-8 (1986); Elvin et al., Eur. J. Immunol. 21(9):2025-31 (1991)), or a conformationally-specific, pan-class II antibody such as LB3.1 to detect properly refolded MHC II molecules (Gorga et al., Cell Immunol. 1986 November;103(1):160-73). Standard refolding conditions yield 1-10 microgram/ml for a variety of peptides and MHC proteins. Alternately, the refolding mixture can contain pre-biotinylated beta-2-microglobulin, with in situ streptavidin capture and direct in-plate W6/32 detection.

Preparation of MHC-Peptide Complexes

MHC-peptide complexes can be produced using any method known in the art, including that described in Frayser et al., supra. Briefly, the method can include incubating the purified, properly folded MHC with a large excess of peptides at low pH, and purifying the MHC-peptide complexes using known methods. In some embodiments, the MHCs are oligomerized, e.g., prior to or after exposure to peptides. The MHCs can be oligomerized using any method known in the art, including by biotinylating the MHC monomers and exposing the MHCs to streptavidin. See, e.g., Altman et al., Science 274(5284):94-6 (1996); Cochran and Stern, Chem. Biol. 7(9):683-96 (2000); Cochran et al., Trends Biochem Sci 26(5):304-10 (2001); and Cochran et al., J. Biol. Chem. 276(30):28068-74 (2001). Oligomerized MHC molecules can also be obtained commercially, e.g., from ProImmune, Springfield, Va.

Antigen-Derived Peptide Selection

In some embodiments, the invention includes arrays that include all possible peptide epitopes, e.g., for one or more protein antigens, e.g., viral or bacterial proteins, e.g., or all of the proteins coded for by a given viral genome. For example, for class II MHC, 10-residue-overlapping 20-mers can be used, corresponding to 25-100 peptides per typical viral protein of 25-100 kdal (˜250-1000 residues), or <1000 per typical viral genome. For class I MHC produced in E. coli, the MHC can be refolded in the presence of each peptide as a mixture of 8, 9, 10, and 11-mer N-terminal variants (e.g., prepared using methods known in the art, e.g., synthesized in a pool, e.g., using a partial tBOC-ON capping strategy (Feng et al., Chem Biol. 3(8):661-70 (1996)) expecting that ˜10% will give measurable refolding, leading to similar numbers of peptides to be evaluated as MHC-peptide complexes. In this way, the methods described herein can be used to identify antigenic epitopes in a known antigen. Fewer than all possible peptides can also be screened. For example, predicted antigenic and/or MHC-binding motifs, and/or catalogs of known or predicted MHC-binding peptides, can be used to reduce the number of peptides to be screened.

In some embodiments, to identify peptides that bind to class II MHC proteins, overlapping peptides can be synthesized so that every 10-mer is present internally on at least one peptide; typically 25-mer peptides with 10-residue overlaps can be used as a balance between synthesis cost and yield constraints. For identifying peptides that bind to class I MHC proteins, every potential peptide must be present as an individual species, and 8, 9, 10, and 11-mer versions of each sequence will be tested. These versions can be synthesized together in a single pot synthesis by a fractional protection strategy.

Immobilization Chemistries

The MHC-peptide complexes can be immobilized on a substrate via the MHC molecules, which can be immobilized on the substrate via direct adsorption, peptide linkers, disulfide, maleimide, vinyl sulfone, or haloacetyl chemistries. Alternatively, the MHC-peptide complexes can be immobilized on the substrate via the antigen-derived peptides, which are immobilized on the surface via direct adsorption, peptide linkers, disulfide, maleimide, vinyl sulfone, or haloacetyl chemistries

MHC proteins can be immobilized onto plastic, glass, or gold surfaces by direct absorption or by C-terminal tagging strategies. For example, C-terminal cysteine residues can be and have been introduced into many MHC proteins using standard techniques of molecular biology, allowing the modified MHC proteins to be attached directly to gold surfaces, or indirectly to suitably modified glass or plastic surfaces, e.g., surfaces modified by introduction of disulfide, maleimide, vinyl sulfone, and/or haloacetyl chemistries. Such surfaces could be, for example, the surface of slides, coverslips, beads, or quantum dots. In one embodiment, biotin-mediated immobilization to streptavidin-coated surfaces can be used to take advantage of the large numbers of recombinant MHC proteins already available in biotinylated form as a result of current efforts to produce MHC proteins for incorporation into streptavidin-linked tetramers.

In addition, instead of immobilizing the MHC, the antigen-derived peptides can be immobilized through similar chemistries, e.g., using a long, flexible linker, and soluble MHC proteins can be bound to the peptides in such a way that T cells can access the relevant binding surface. A variety of costimulatory molecules, e.g., costimulatory antibodies (e.g., anti-CD2, anti-CD11a, anti-CD28 and/or anti-CD49d) and/or purified costimulatory agents (e.g., B7-1 (also known as CD80) B7-2 (also known as CD86), ICOSL (also known as B7h, LICOS, B7-H2, or B7RP-1), B7-H1 (also known as PD-L1), B7-DC (also known as PD-L2), B7-H3, B7-H4, or other member of the B7 family (see, e.g., Rietz and Chen, (2004) Am. J. Transpl. 4:8-14), LFA-3, and/or ICAM-1, ICAM-2, or other members of the ICAM family) can be similarly immobilized. Other immobilization chemistries can also be used, e.g., as described in U.S. Pat. Nos. 6,436,647 or 6,319,674. In some embodiments, the peptides are synthesized directly on the chip (see, e.g., Gao et al., Proteomics. 3(11):2135-41 (2003); Frank, J. Immunol. Methods. 1;267(1):13-26 (2002); Pellois et al., Nat Biotechnol. 20(9):922-6 (2002); Pipkorn et al., J Pept Res. 59(3):105-14 (2002).

Spotting MHC-Peptide Complexes onto Substrates

MHC-peptide complexes can be applied to substrates by automated spotting robots (arrayers) or by manual application of small drops to the substrate in an array format. For manual application, approximately 10 to 1000 nanoliter drops are applied by hand using a pipettor, and allowed to dry at ambient temperature. For automated application, 1 to 100 nanoliter drops can be applied by pin-and-ring, split-pin, or other application technologies. A number of automated spotter apparatuses, which can be modified for use in the methods described herein, are commercially available, including the Generation III Array Spotter (Amersham), the Lucidea™ Array Spotter (Amersham), the FlexSys™ Robotic Array Spotter (Genomics Solutions), and the Affymetrix 417™ Arrayer (Affymetrix), among others. A number of U.S. patents describe methods and devices for spotting solutions onto substrates, including U.S. Pat. Nos. 6,447,723 and 5,763,170, inter alia.

Assays for Identifying Positive Responses

A number of different methods can be used to detect a positive response, i.e., specific binding of a T cell to an MHC-peptide complex, including, but not limited to, those methods illustrated in FIG. 3, e.g., detecting intracellular signals such as calcium flux or phosphorylation, downregulation of T cell receptor complex, upregulation of activation markers on the cell surface such as CD69, CD25, and/or CD71, and secretion of factors such as IL2 and IFN; any of these, or any other detectable response, can serve as a readout for identifying positive responses. Any method of detection known in the art can be used, including microscopy, imaging, micromechanics (U.S. Pat. No. 6,436,647), or observation. Data can be collected manually or automatically, e.g., using a chip reader or flow cytometer as known in the art.

T Cell Adhesion Assay

As shown in FIG. 3A, after incubation, the arrays can be washed to remove any non-adhering T cells, and known methods can be used to visualize the response, e.g., Nomarski or phase contrast microscopy. For example, the cells can be fixed and incubated with fluorescent antibodies, colloidal gold, or precipitating enzyme substrates to detect the cells. Surface plasmon resonance technologies can also be used. In this embodiment, mere T cell binding can be detected, in addition to or alternative to detecting activation.

T Cell Upregulation Assay

As shown in FIG. 3B, after incubation, cells can be fixed and incubated, e.g., with fluorescent antibodies to label surface markers, e.g., constitutive (e.g., CD2, CD3, CD4, CD5, CD7, and CD8) and activation-induced (e.g., CD25, CD44, CD69 and CD154) T cell surface markers. Alternatively, colloidal gold or precipitating enzyme substrates can provide an alternative labeling method. Standard microscopy methods, e.g., phase-contrast or fluorescence microscopy, can be used to detect the label.

As one example, in a variety of CD4+ and CD8+ T cells, consistent significant upregulation of CD25 and CD69 (e.g., from very low basal levels to >10,000/cell) has been observed in response to immobilized MHC-peptide complexes. Thus, these activation markers, CD3 downregulation, and intracellular cytokine staining can be used for detecting of productive T cell/MHC-peptide interaction (FIGS. 2A-D). Methods known in the art, including the methods described in Cochran et al., Immunity, 12:241-250 (2000), can be used to detect cell surface marker upregulation. Intracellular cytokine staining can be measured, e.g., using methods known in the art.

Ca⁺⁺ Flux Assay

As shown in FIG. 3C, intracellular signals, such as fluctuations in calcium concentrations, can also be used to detect T cell activation, using methods known in the art, such as those described in Billups et al., Eur. J. Physiol. 444:663-669 (2002). Briefly, prior to or during incubation on the array, T cells can be loaded with a calcium-sensitive dye, e.g., a fluorescent calcium-sensitive dye, e.g., fura-2, fluo-3, calcium crimson, calcium green, or Oregon green, before exposure to immobilized MHC-peptide complexes, and standard imaging methods, e.g., fluorescence video microscopy, can be used to follow fluctuations in Ca⁺⁺ concentrations, e.g., transients.

Secretion Assay

As shown in FIG. 3D, antibodies, e.g., antibodies specific for cytokines or other secreted factors, can be pre-immobilized on slides or co-immobilized with MHC-peptide complexes, and used to capture factors secreted by activated T cells. After incubation the slides can be washed and developed with a second antibody specific for cytokines or other secreted factors, e.g., a labeled second antibody, similar to an ELISA or ELISPOT assay. Preferably, the second antibody will bind the cytokine or other factor at a different epitope from the immobilized antibody, and neither will interfere with the binding of the other.

The second antibody can be labeled with a detectable moiety, e.g., a radioactive moiety (e.g., ³⁵S, ³²P, ³H, or ¹⁴C), a chemiluminescent moiety (e.g., Streptavidin-Alkaline Phosphatase, Streptavidin-Horseradish Peroxidase, Streptavidin-Biotinylated Horseradish Peroxidase, e.g., for detection with ECLTM or a variant thereof (Amersham Biosciences, Piscataway, NJ)), a fluorescent moiety (e.g., CyDyes (such as Cy3, Cy3B, Cy3.5, Cy5, Cy5.5, Cy7, Cy5Q, Cy7Q, Cy2-Streptavidin, Cy3-Streptavidin, Cy5-Streptavidin, Streptavidin-Fluorescein, Streptavidin-Texas Red (Amersham Biosciences, Piscataway, N.J.), fluorescein, rhodamine, Texas red, cyanine, Cascade Blue, or phycoerythrin), quantum dots (see, e.g., Watson et al., BioTechniques February, 2003; 34(2):296-300, 302-3; Goldman et al., J. Am. Chem. Soc. Jun. 5, 2002;124(22):6378-82; Han et al., Nat. Biotechnol. July, 2001;19(7):631-5; Chan et al., Science Sep. 25, 1998 281(5385):2016-8), or other directly or indirectly detectable moiety (e.g., gold or other particles). These moieties can be detected using methods known in the art. For example, a number of methods are known in the art for detection of fluorescent moieties, including, but not limited to, fluorescence polarization (FP), fluorescence resonance energy transfer (FRET), time-resolved fluorescence resonance energy transfer (TR-FRET), and fluorescence intensity (FI).

Multiple secreted factors can be assayed concurrently, e.g., using second antibodies with different labels, e.g., labeled with multicolored fluorescent moieties or quantum dots. Thus, multiple different factors and/or activation markers can be simultaneously assayed; for example, antibodies to different factors can be associated with quantum dots or fluorescent compounds with different fluorescence properties. Other soluble factors secreted by T cells in addition to cytokines, such as alpha-defensin, can be detected similarly.

### EXAMPLES

The invention is further described in the following examples, which do not limit the scope of the invention described in the claims.

The following materials were used in the examples described below. PBST was 1×PBS; 0.05% Triton X100; filtered and stored at 4° C. The “T cell medium” (for assays) contained 10% Fetal Bovine Serum, heat inactivated; 1:100 Penn-Strep solution (Gibco); 1:100 Glutamine solution (Gibco); 1:100 Hepes solution (Gibco); Fill with 1×RPMI with phenol red (Gibco); and was filtered and stored at 4° C. A “Dilution Solution” was 1×PBS; 0.3% BSA; 0.1% Triton X-100; and was filtered and stored at 4° C. The cytokine capture antibody and detection antibody, Avidin-HRP, and AEC Substrate set were commercially available reagents, obtained from B.D. Biosciences Pharmingen, San Diego, Calif.; streptavidin-Alexa 647 was obtained from Molecular Probes, Eugene, Oreg.; and the LB3.1-Cy-3 was produced by purifying the antibody from hybridoma supernatants and then labeling using an amine-reactive Cy-3 reagent from Molecular Probes, Eugene, Oreg. Other reagents, e.g., homemade reagents can also be used.

### Example 1

### Making Cytokine Capture MHC-Peptide Array Chips

To make a cytokine capture MHC-peptide array, 25 mm×75 mm polystyrene slide was marked with an array pattern on the back using a pre-printed template, leaving the frosted/rough handle area free. The array area on the front of the slide was then surrounded with a hydrophobic barrier using hydrophobic ink or a silicone gasket. The hydrophobic ink around the array area was then allowed to dry. The slide was placed into a secondary container, typically a petri dish, to catch any spills.

Monoclonal cytokine capture antibody (not biotinylated) was diluted 1:200 in PBS to make 1.0-1.5 mL of diluted antibody solution per slide, depending on the hydrophobic barrier used (1.0 mL for hydrophobic ink, 1.5 mL for 0.5 mm thick silicone gasket). The diluted antibody solution was spread onto the array area of the slide, making sure it was completely covered, and there was no leaking outside the hydrophobic barrier.

The antibody solution was incubated on the slide overnight at 4° C., then the antibody solution was removed and the slide was allowed to dry.

MHC and/or antibody solutions were spotted onto the slide in a pre-selected pattern, using an arraying robot, or by hand, guided by the template on the back surface of the slide. For hand-spotted arrays, 0.5 μL of solution was used per spot. Anti-CD3 was used as a control in some spots, and spots of streptavidin were used as a development control as it develops even if the T cells do not respond. The anti-CD3 or MHC-peptide complex were combined with a co-stimulatory antibody such as anti-CD11a, anti-CD2, anti-CD28 and/or anti-CD49d to enhance the activation without causing activation by non-specific peptide-MHC complexes.

Concentrations were 1 μM or approximately 50 μg/mL of MHC-peptide complex, and/or 5 μg/mL of antibody solution. Alternatively, the MHC-peptide complexes were tetramerized by biotinylating the soluble monomers and adding them to streptavidin, keeping the concentration of MHC at 50 μg/mL. The spots were then allowed to dry completely.

The slide was then labeled with the capture antibody used, an indication of the antigen presentation pattern spotted, and the date completed. The array slide was stored at 4° C. in a desiccator until used.

### Example 2

### T-Cell Epitope Detection Experiment Using Modified Cytokine Capture MHC-Peptide Array

To determine whether a cytokine capture MHC-peptide array as described herein could be used to detect T-cell epitopes, an appropriate 25 mm×75 mm MHC-peptide array on a polystyrene slide was placed inside a petri dish if it was not already in one. About 30 minutes before the T cell sample was to be placed on the slide, 1.0-1.5 mL of T cell medium was spread inside the hydrophobic barrier making sure the entire area is covered, and the array was allowed to sit at room temperature. This served to block the array.

The T cell sample was resuspended and counted. Enough T cells were added so that at least about 1×10⁴ specific cells would be present on each array in 1.0-1.5 mL T cell medium. An appropriate amount of the T cell solution was centrifuged at 1500 RPM for 10 minutes at 4° C. to spin down the cells, and the supernatant was aspirated. The T cell pellet was resuspended in 1.0-1.5 mL T cell medium. Five ng/mL PMA was used to enhance the antigen-specific activation; PMA alone does not cause activation in the T cells.

The T cell medium that was blocking the surface was then aspirated from the MHC-peptide array. The freshly re-suspended T cells in medium were added to the surface of the array inside the hydrophobic barrier, making sure the entire area was covered.

The array with the T cells was then placed inside a well-humidified 5% CO₂, 37° C. incubator, being careful not to spill the T cell solution, and making sure that the array was level inside the incubator. The array was incubated at 37° C., 5% CO₂ for 4-48 hours, depending on the factor to be analyzed and the type of T cell (CD4⁺ or CD8⁺, murine or human, etc.).

After incubation, the array was removed from the incubator, and the cell solution was aspirated off. ddH₂O was spread inside the hydrophobic barrier, and the array was allowed to sit for 5 minutes at room temperature, to lyse any remaining cells. The 5 minute ddH₂O wash was then repeated. Next, the ddH₂O was removed and the slide was washed 3 times with PBST, aspirating the buffer off after each wash.

Monoclonal cytokine detection antibody (biotinylated) diluted 1:250 in Dilution Solution was used to coat the array inside the hydrophobic barrier (1.0-1.5 mL/array). The array was incubated with the detection antibody solution for 2 hours at room temperature, then washed 3 times with PBST.

Avidin-HRP stock solution diluted 1:100 in Dilution Solution was then used to coat the array inside the hydrophobic barrier (1.0-1.5 mL/array). The array was incubated with the Avidin-HRP solution for 1 hour at room temperature, then washed 3 times with PBST, then washed 2 additional times with azide-free PBS, as azide will inhibit the HRP enzyme.

1.0-1.5 mL of the AEC substrate solution (the AEC substrate solution was prepared no more than fifteen minutes before use by adding 20 μL of substrate per milliliter of buffer solution and mixing well) was spread inside the hydrophobic barrier on the array, making sure the entire area is covered. This reaction was allowed to proceed in the dark for 5-60 minutes, the progress was checked periodically.

To stop the reaction, the AEC solution was aspirated off and the array was washed 2 times with ddH₂O and allowed to dry completely in the dark.

The arrays were observed under a dissection microscope, and precipitated spots were detected only in areas that carried MHC-peptide complexes specific for the T cells (see FIG. 6, right panel), and not in areas that carried non-specific MHC-peptide complexes (see FIG. 6, middle panel).

These results demonstrate that a cytokine capture MHC-peptide array, e.g., as described herein, can be used to detect specific T-cell epitopes chromogenically.

### Example 3

### T-Cell Epitope Detection Experiment Using Modified Cytokine Capture MHC-Peptide Array with Fluorescent Detection

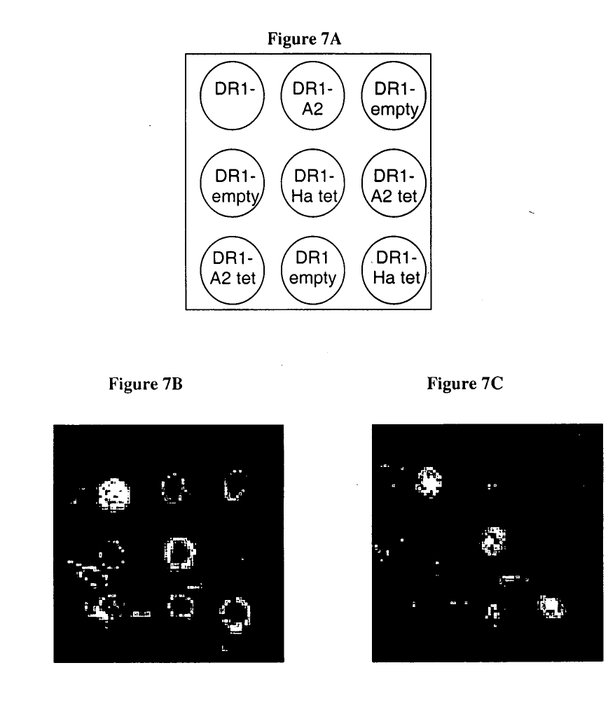

To determine whether a cytokine capture MHC-peptide array as described herein could be used to detect T-cell epitopes using fluorescent methodology, which would be more appropriate for high throughput screening methods, an appropriate 25 mm×75 mm MHC-peptide array on a polystyrene-coated glass slide was placed inside a petri dish if it was not already in one. Anti-mouse IL2 capture antibody was co-immobilized in the 0.5 microliter spots as shown in FIG. 7A. About 30 minutes before the T cell sample was to be placed on the slide, 1.0-1.5 mL of T cell medium was spread inside the hydrophobic barrier making sure the entire area is covered, and the array was allowed to sit at room temperature. This served to block the array.

The T cell sample was resuspended and counted. 1×106 HA1.7 T cell hybridomas (specific for DR1-Ha complex; Xu and Littman, Cell 74:633 (1993); Boen et al., J. Immunol. 165:2040-2047 (2000)) were incubated on the chip for 16 hours at 37 degrees C., 5% CO2. Enough T cells were added so that at least about 1×104 specific cells would be present on each array in 1.0-1.5 mL T cell medium. An appropriate amount of the T cell solution was centrifuged at 1500 RPM for 10 minutes at 4° C. to spin down the cells, and the supernatant was aspirated. The T cell pellet was resuspended in 1.0-1.5 mL T cell medium.

The T cell medium that was blocking the surface was then aspirated from the MHC-peptide array. The freshly re-suspended T cells in medium were added to the surface of the array inside the hydrophobic barrier, making sure the entire area was covered.

The array with the T cells was then placed inside a well-humidified 5% CO₂, 37° C. incubator, being careful not to spill the T cell solution, and making sure that the array was level inside the incubator. The array was incubated at 37° C., 5% CO₂ for 4-48 hours (typically around 16 hours), depending on the factor to be analyzed and the type of T cell (e.g., CD4⁺ or CD8⁺, murine or human).

After incubation, the array was removed from the incubator, and the cell solution was aspirated off. ddH₂O was spread inside the hydrophobic barrier, and the array was allowed to sit for 5 minutes at room temperature, to lyse any remaining cells. The 5 minute ddH₂O wash was then repeated. Next, the ddH₂O was removed and the slide was washed 3 times with PBST, aspirating the buffer off after each wash.

Monoclonal cytokine detection antibody (biotinylated, enough for 1:250 final dilution) was pre-incubated for 15-30 minutes with streptavidin fluorescently labeled with Alex647 (enough for 1:1000 final dilution). After pre-incubation, the mixture was diluted Dilution Solution containing an extra 100 micromolar concentration of D-biotin to block non-specific biotin-streptavidin interaction with tetramers in the MHC array. Fluorescently-labeled anti-MHC antibody (Cy-3) was added to the diluted mixture for a final concentration of 1:1000. This mixture was used to coat the array inside the hydrophobic barrier (1.0-1.5 mL/array). The array was incubated with the detection antibody solution for 2 hours in the dark at room temperature, then washed 3 times with PBST.

After the PBST washes, the slide was washed two more times with ddH₂O to remove salt and surfactant. The slides were dried, and the hydrophobic barrier was removed (when appropriate). The dry slides were stored in the dark at room temperature until analyzed.

The slide was blocked after the spots dried, the chip was washed, and then simultaneously stained with LB3.1-Cy3 and pre-incubated biotinylated anti-mouse IL2 and streptavidin-Alexa 647.

The arrays were observed using an Affymetrix array scanner detecting both Cy-3 and Alexa 647 fluorescence (Alexa 647 emits at the same wavelengths as Cy-5). The spots that show Cy-3 fluorescence indicate native-form HLA-DR1 complex (see FIG. 7B), and spots which show Cy-5 fluorescence indicate captured and detected factors secreted by the T cells (see FIG. 7C).

These results demonstrate that a cytokine capture MHC-peptide array, e.g., as described herein, can be used to detect specific T-cell epitopes using fluorescence.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An array comprising a substrate and an array of MHC molecules complexed with antigen-derived peptides immobilized in spatially-distinct areas on the substrate.

2. The array of claim 1, wherein the MHC molecules in all of the spatially-distinct areas are the same.

3. The array of claim 1, wherein the spatially-distinct areas are surrounded by a hydrophobic barrier.

4. The array of claim 1, wherein the spatially-distinct areas are each surrounded by a hydrophobic barrier.

5. The array of claim 1, wherein the array comprises at least about 10, 50, or 100 different MHC-peptide complexes.

6. The array of claim 1, wherein the substrate is optically transparent.

7. The array of claim 1, wherein the substrate comprises glass, quartz, polystyrene, polycarbonate, polypropylene, polymethacrylate, or silicon.

8. The array of claim 1, wherein the substrate is coated with gold, biotin streptavidin, or another molecule used to immobilize the MHC molecules.

9. The array of claim 1, wherein the MHC-peptide complexes are immobilized on the substrate via the MHC molecule, which is immobilized via direct adsorption, peptide linkers, biotin-streptavidin; cysteine attachment; amine attachment; or metal chelate interaction.

10. The array of claim 1, wherein the MHC-peptide complexes are immobilized on the substrate via the antigen-derived peptide, which is immobilized via direct adsorption; peptide linkers; biotin-streptavidin; cysteine attachment; amine attachment; or metal chelate interaction.

11. An array comprising a substrate and an array of MHC molecules complexed with antigen-derived peptides and costimulatory molecules immobilized in spatially-distinct areas on the substrate.

12. The array of claim 11, wherein the costimulatory molecules are selected from the group consisting of costimulatory antibodies and costimulatory agents.

13. The array of claim 12, wherein the costimulatory antibodies bind specifically to one or more of CD2, CD11a, CD28, or CD49d.

14. The array of claim 11, wherein the costimulatory agent is B7-1, B7-2, ICOSL, B7-H1, B7-DC, B7-H3, B7-H4, LFA-3, ICAM-1, or ICAM-2.

15. The array of claim 11, wherein the array comprises at least about 10, 50, or 100 different MHC-peptide complexes.

16. The array of claim 11, wherein the MHC molecules comprise Class I MHC molecules, Class II MHC molecules, or Class I and Class II MHC molecules.

17. The array of claim 11, wherein the MHC-peptide complexes are immobilized on the substrate via the MHC molecule, which is immobilized via direct adsorption, peptide linkers, biotin-streptavidin; cysteine attachment; amine attachment; or metal chelate interaction.

18. The array of claim 11, wherein the MHC-peptide complexes are immobilized on the substrate via the antigen-derived peptide, which is immobilized via direct adsorption; peptide linkers; biotin-streptavidin; cysteine attachment; amine attachment; or metal chelate interaction.

19. An array comprising a substrate and an array of MHC molecules complexed with antigen-derived peptides, and anti-factor antibodies specific for secreted factors, immobilized in spatially-distinct areas on the substrate.

20. The array of claim 19, wherein the immobilized anti-factor antibodies bind specifically to one or more of IL-2, IL-3, IL-4, IL-5, IL-6, IL-7, IL-9, IL10, IL-12, IL-13, IL-16, interferon-gamma (IFN-γ), tumor necrosis factor alpha (TNF-α), tumor necrosis factor beta (TNF-β), GM-CSF, oncostatin M (OSM), macrophage migration inhibitory factor (MIF), TNF-Related Apoptosis Inducing Ligand (TRAIL), 4-1BB ligand (4-1BBL), or alpha-defensin.

21. The array of claim 18, wherein the array comprises at least about 10, 50, or 100 different MHC-peptide complexes.

22. The array of claim 18, wherein the MHC molecules comprise Class I MHC molecules, Class II MHC molecules, or Class I and Class II MHC molecules.

23. The array of claim 18, wherein the MHC-peptide complexes are immobilized on the substrate via the MHC molecule, which is immobilized via direct adsorption, peptide linkers, biotin-streptavidin; cysteine attachment; amine attachment; or metal chelate interaction.

24. The array of claim 18, wherein the MHC-peptide complexes are immobilized on the substrate via the antigen-derived peptide, which is immobilized via direct adsorption; peptide linkers; biotin-streptavidin; cysteine attachment; amine attachment; or metal chelate interaction.

25. The array of claim 11, further comprising anti-factor antibodies specific for secreted factors immobilized in spatially-distinct areas on the substrate.

26. A method for identifying a T cell epitope, the method comprising:
providing an array comprising a substrate and an array of MHC molecules complexed with antigen-derived peptides immobilized in spatially-distinct areas on the substrate;
contacting the array with a sample comprising T cells;
detecting a T cell interaction with an MHC-peptide complex; and
identifying the T cell epitope based on the identity of the MHC-peptide complex.

27. The method of claim 26, wherein the interaction is detected by detecting activation of T cells by one or more of factor secretion, expression of an activation marker, or an intracellular signal.

28. The method of claim 27, wherein the intracellular signal is calcium flux.

29. The method of claim 27, wherein the activation marker is CD3, CD4, CD8, Cd11a, CD25, CD27, CD28, CD44, CD49e, CD62L, CD69, CD71, CD95, CD152, or Ly6A.

30. The method of claim 27, wherein the secreted factor is IL-2, IL-3, IL-4, IL-5, IL-6, IL-7, IL-9, IL10, IL-12, IL-13, IL-16, interferon-gamma, tumor necrosis factor alpha, TNF-b, GM-CSF, oncostatin M, macrophage migration inhibitory factor, TNF-Related Apoptosis Inducing Ligand, 4-1BB ligand, α-defensin, or CD40 ligand.

31. The method of claim 26, wherein the interaction is detected by detecting expression of CD40 ligand, CD30 ligand, CD27 ligand, or Fas ligand.

32. The method of claim 26, wherein the array further comprises immobilized anti-factor antibodies, and factor secretion is detected by detecting binding of a factor to an immobilized anti-factor antibody.

33. The method of claim 32, wherein the factor is IL-2, IL-3, IL-4, IL-5, IL-6, IL-7, IL-9, IL-10, IL-12, IL-13, IL-16, interferon-gamma, tumor necrosis factor alpha, TNF-b, GM-CSF, oncostatin M, macrophage migration inhibitory factor, TNF-Related Apoptosis Inducing Ligand, 4-1BB ligand, or α-defensin.

34. A method of making an array, the method comprising providing a substrate, and immobilizing MHC molecules complexed with antigen-derived peptides in spatially-distinct areas on the substrate.

35. The method of claim 34, wherein the MHC molecules in all of the spatially-distinct areas are the same.

36. The method of claim 34, further comprising surrounding the spatially-distinct areas with a hydrophobic barrier.

37. The method of claim 34, further comprising surrounding each one of the spatially-distinct areas with a hydrophobic barrier.

38. The method of claim 34, wherein the array comprises at least about 10, 50, or 100 different MHC-peptide complexes.

39. The method of claim 34, wherein the MHC molecules comprise Class I MHC molecules, Class II MHC molecules, or Class I and Class II MHC molecules.

40. The method of claim 34, wherein the substrate is optically transparent.

41. The method of claim 34, wherein the substrate comprises glass, quartz, polystyrene, polycarbonate, polypropylene, polymethacrylate, or silicon.

42. The method of claim 34, wherein the substrate is coated with gold, biotin streptavidin, or another molecule used to immobilize the MHC molecules.

43. The method of claim 34, wherein the MHC-peptide complexes are immobilized on the substrate via the MHC molecule, which is immobilized via direct adsorption, peptide linkers, biotin-streptavidin; cysteine attachment; amine attachment; or metal chelate interaction.

44. The method of claim 34, wherein the MHC-peptide complexes are immobilized on the substrate via the antigen-derived peptide, which is immobilized via direct adsorption; peptide linkers; biotin-streptavidin; cysteine attachment; amine attachment; or metal chelate interaction.

45. The method of claim 34, further comprising immobilizing costimulatory molecules on the substrate.

46. The method of claim 45, wherein the costimulatory molecules are selected from the group consisting of costimulatory antibodies and costimulatory agents.

47. The method of claim 46, wherein the costimulatory antibodies are one or more of anti-CD2, anti-CD11a, anti-CD28, or anti-CD49d.

48. The method of claim 46, wherein the costimulatory agent is B7-1, B7-2, ICOSL, B7-H1, B7-DC, B7-H3, B7-H4, LFA-3, ICAM-1, or ICAM-2.

49. The method of claim 34, further comprising immobilizing anti-factor antibodies specific for secreted factors on the substrate.

50. The method of claim 49, wherein the immobilized anti-factor antibodies comprise at least about one of antibodies specific for IL-2, IL-3, IL-4, IL-5, IL-6, IL-7, IL-9, IL-10, IL-12, IL-13, IL-16, IFN-γ, TNF-α, TNF-β, GM-CSF, oncostatin M, macrophage migration inhibitory factor, TNF-Related Apoptosis Inducing Ligand, 4-1BB ligand, or alpha-defensin.

51. The method of claim 45, further comprising immobilizing anti-factor antibodies specific for secreted factors on the substrate.

52. The method of claim 51, wherein the immobilized anti-factor antibodies comprise at least about one of antibodies specific for IL-2, IL-3, IL-4, IL-5, IL-6, IL-7, IL-9, IL-10, IL-12, IL-13, IL-16, IFN-γ, TNF-α, TNF-β, GM-CSF, oncostatin M, macrophage migration inhibitory factor, TNF-Related Apoptosis Inducing Ligand, 4-1BB ligand, or alpha-defensin.
